(19) United States
(12) Patent Application Publication
(10) Pub. No.: US 2023/0226347 A1
(43) Pub. Date: Jul. 20, 2023

(54) MEDICAL IMPLANT WITH PRE-SHAPED ASSEMBLY

(71) Applicant: Cochlear Limited, Macquarie University, NSW (AU)

(72) Inventors: Nicholas Charles Pawsey, Macquarie University (AU); Shahram Manouchehri, Macquarie University (AU)

(21) Appl. No.: 18/002,414

(22) PCT Filed: Jun. 7, 2021
(86) PCT No.: PCT/IB2021/054994

§ 371 Date: Dec. 19, 2022

Related U.S. Application Data
(60) Provisional application No. 63/054,043, filed on Jul. 20, 2020.

(51) Int. Cl. A61N 1/05; A61N 1/36

(52) U.S. Cl. CPC A61N 1/0541; A61N 1/36038; A61N 1/375

(57) ABSTRACT

An apparatus includes a container and a medical device configured to be implanted on or within the recipient's body. The medical device is contained within a sealed region of the container prior to implantation of the medical device. The medical device includes at least one housing containing circuitry and a plurality of signal ports spaced from the at least one housing. The plurality of signal ports is configured to be in communication with a portion of the recipient's body. The medical device further includes an elongate assembly extending from the at least one housing and configured to transmit signals between the circuitry and the plurality of signal ports. The assembly has at least one portion having a shape while the medical device is within the sealed region, the shape including at least one loop and/or a plurality of serpentine turns.

### BACKGROUND

### Field

The present application relates generally to systems and methods for facilitating implantation of a medical device on or within a recipient's body, and more specifically to cochlear implant auditory prostheses.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a container comprising a sealed region and a medical device configured to be implanted on or within the recipient's body. The medical device is contained within the sealed region prior to implantation of the medical device. The medical device comprises at least one housing containing circuitry and a plurality of signal ports spaced from the at least one housing. The plurality of signal ports is configured to be in communication with a portion of the recipient's body. The medical device further comprises an elongate assembly extending from the at least one housing and configured to transmit signals between the circuitry and the plurality of signal ports. The assembly has at least one portion having a shape while the medical device is within the sealed region, the shape comprising at least one loop and/or a plurality of serpentine turns.

In another aspect disclosed herein, a method comprises accessing a medical implant comprising at least one portion having a pre-formed coil shape with a first number of coil turns, the first number greater than or equal to one. The method further comprises modifying the at least one portion to have a modified coil shape with a second number of coil turns, the second number equal to the first number. The method further comprises implanting the medical implant on or within a recipient's body with the at least one portion having the modified coil shape.

In another aspect disclosed herein, a method comprises placing a plurality of substantially straight signal conduits into a substantially straight tube and inserting a material into the tube. The method further comprises manipulating the tube containing the plurality of signal conduits and the material into a helix shape or a serpentine shape about a mandrel. The method further comprises curing the material and removing the tube from the mandrel.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 schematically illustrates a simplified side view of an example internal component;

FIG. 3A schematically illustrates an example apparatus in accordance with certain implementations described herein;

FIGS. 3B and 3C illustrate two example apparatus comprising a cochlear implant auditory prosthesis in accordance with certain implementations described herein;

FIGS. 4A and 4B schematically illustrate a perspective view and a top view, respectively, of the example medical device of FIG. 3B on an anatomical mock-up to show the medical device after implantation in accordance with certain implementations described herein;

FIG. 5 is a flow diagram of an example method in accordance with certain implementations described herein; and

FIG. 6 is a flow diagram of an example method in accordance with certain implementations described herein.

### DETAILED DESCRIPTION

Implantable medical devices (e.g., cochlear implants) have leads that are longer than the typical distance between the site of the controlling circuitry (e.g., the receiver/stimulator) and the portion of the recipient's body (e.g., cochlea) being stimulated or monitored, in order to accommodate variations in anatomy among different recipients, surgical preferences, and/or growth (e.g., skull growth) for pediatric recipients. For cochlear implants, this excess lead length is typically secured within the mastoid cavity following insertion of the stimulation assembly into the cochlea. However, the manipulation of the lead during this process can transmit movement to the stimulation assembly within the cochlea, which can result in trauma and/or compromise the preservation of acoustic hearing. Furthermore, improper securing of the excess lead length can result in migration of the stimulation assembly out of the cochlea after surgical implantation, to be replaced by a subsequent surgical procedure.

Certain implementations described herein provide a pre-shaped portion configured to avoid or reduce the manipulation of excess lead length during the initial implantation and to prevent or reduce the probability of unwanted movement after implantation (e.g., due to migration or growth of the recipient). The pre-shaped portion can comprise at least one malleable element configured to maintain the shape of the pre-shaped portion, to allow for selective modification (e.g., extending and/or bending without changing a number of loops or serpentine turns) of the shape by the user before or during implantation of the medical device, and to maintain the user-modified shape after implantation.

The teachings detailed herein are applicable, in at least some implementations, to any type of implantable medical device (e.g., implantable sensory prostheses) configured to apply stimulation signals to a portion of the recipient's body. The implanatable medical device of certain implementations described herein comprises a first portion (e.g., external to a recipient) and a second portion (e.g., implanted on or within the recipient), the first portion configured to wirelessly transmit power and/or data to the second portion. For example, the implantable medical device can comprise an auditory prosthesis system utilizing an external sound processor configured to transcutaneously provide power to an implanted assembly (e.g., comprising an actuator). In certain such examples, the external sound processor is further configured to transcutaneously provide data (e.g., control signals) to the implanted assembly that responds to the data by generating stimulation signals that are perceived by the recipient as sounds. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. The concepts described herein and/or variations thereof can be applied to any of a variety of implantable medical devices comprising an implanted component configured to use magnetic induction to receive power (e.g., transcutaneously) from an external component and to store at least a portion of the power in at least one power storage device (e.g., battery). The implanted component can also be configured to receive control signals from the external component (e.g., transcutaneously) and/or to transmit sensor signals to the external component (e.g., transcutaneously) while receiving power from the external component.

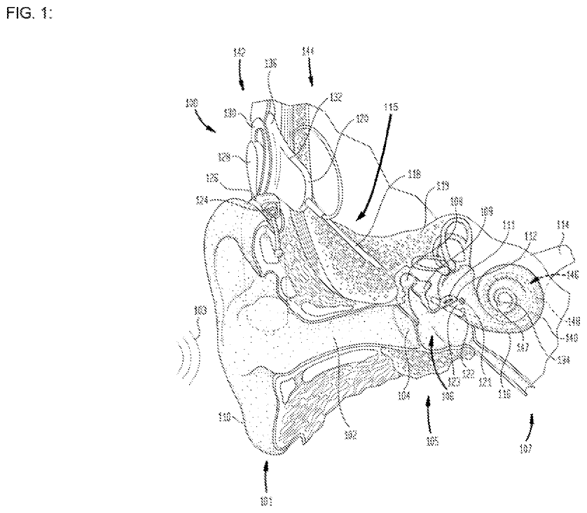

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

The human skull is formed from a number of different bones that support various anatomical features. Illustrated in FIG. 1 is the temporal bone 115 which is situated at the side and base of the recipient's skull (covered by a portion of the recipient's skin/muscle/fat, collectively referred to herein as tissue). For ease of reference, the temporal bone 115 is referred to herein as having a superior portion and a mastoid portion. The superior portion comprises the section of the temporal bone 115 that extends superior to the auricle 110. That is, the superior portion is the section of the temporal bone 115 that forms the side surface of the skull. The mastoid portion, referred to herein simply as the mastoid bone 119, is positioned inferior to the superior portion. The mastoid bone 119 is the section of the temporal bone 115 that surrounds the middle ear 105.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone 115 adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the stimulation elements 148 of the array 146 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

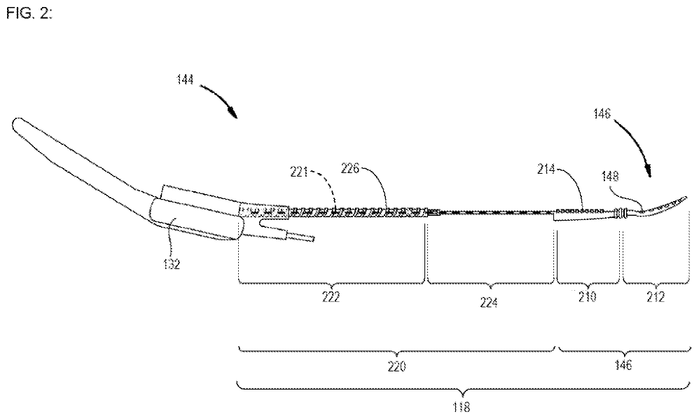

FIG. 2 schematically illustrates a simplified side view of an example internal component 144 comprising an internal receiver unit 132 which receives encoded signals from an external component 142 of the auditory prosthesis 100 (e.g., cochlear implant system). The internal component 144 terminates in the stimulation assembly 118 that comprises an extra-cochlear region 210 and an intra-cochlear region 212. The intra-cochlear region 212 is configured to be implanted in the recipient's cochlea 140 and has disposed thereon the longitudinally aligned and distally extending array 146 (e.g., electrode array; contact array) comprising a plurality of stimulation elements 148. In the example schematically illustrated in FIG. 2, the plurality of stimulation elements 148 comprises electrical contacts (e.g., electrodes) configured to apply electrical stimulation and/or optical contacts (e.g., emitters) configured to apply optical stimulation, either alone or in conjunction with electrical or other stimulation mechanisms.

In certain implementations, the stimulation assembly 118 comprises a lead region 220 coupling the internal receiver unit 132 to the array 146. In certain implementations, optical and/or electrical stimulation signals generated by the internal receiver unit 132 are delivered to the array 146 via the lead region 220. The lead region 220 comprises a first portion 222 configured to accommodate movement (e.g., is flexible) and a second portion 224 configured to connect the first portion 222 to the array 146. The first region 222 of certain implementations is configured to prevent the stimulation assembly 118, the lead region 220 and its connection to the internal receiver unit 132, and the array 146 from being damaged due to movement of the internal component 144 (or part of the internal component 144) which may occur, for example, during mastication. In certain implementations, the second region 224 comprises a distinct connection to the first region 222 and/or the array 146, while in certain other implementations, the second region 224 is blended into the first region 222 and/or the array 146. The relative lengths of the stimulation assembly 118, the lead region 220, the first portion 222, the second portion 224, the extra-cochlear region 210, the intra-cochlear region 212, and the array 146 are not shown to scale in FIG. 2.

In certain implementations, the lead region 220 comprises a body 226 and a plurality of signal conduits (e.g., electrical wire leads; optical waveguides)(not shown) within the body 226. For example, the body 226 can comprise silicone or other biocompatible material in which the signal conduits are embedded (e.g., the body 226 is molded around the signal conduits) or the body 226 can comprise a tube in which the signal conduits are contained (e.g., the tube backfilled with silicone). The signal conduits of certain implementations comprise wires (e.g., platinum; platinum-iridium alloys) having outer diameters that are are wavy or helixed around an axis substantially parallel to the longitudinal direction 221 of the lead region 220 (e.g., within the first region 222) and/or are substantially straight and substantially parallel to the longitudinal direction 221 (e.g., within the second region 224). In certain implementations, each of the signal conduits is connected to a corresponding one of the plurality of stimulation elements 148 of the array 146.

In certain implementations, the extra-cochlear region 210 is located in the middle ear cavity of the recipient after implantation of the intra-cochlear region 212 into the cochlea 140. Thus, the extra-cochlear region 210 corresponds to a middle ear cavity sub-section of the array 146. In certain implementations, an outer surface of the extra-cochlear region 210 comprises nubs 214 configured to aid in the manipulation of the stimulation assembly 118 during insertion of the intra-cochlear region 212 into the cochlea 140.

Various types of stimulation assemblies 118 are compatible with certain implementations described herein, including short, straight, and peri-modiolar. In certain implementations, the stimulation assembly 118 is a peri-modiolar stimulation assembly 118 having an intra-cochlear region 212 that is configured to adopt a curved configuration during and or after implantation into the recipient's cochlea 140. For example, the intra-cochlear region 212 of the stimulation assembly 118 can be pre-curved to the same general curvature of a cochlea 140. Such peri-modiolar stimulation assemblies 118 are typically held straight by, for example, a stiffening stylet (not shown) or sheath which is removed during implantation, or alternatively by varying material combinations or the use of shape memory materials, so that the stimulation assembly 118 can adopt its curved configuration when in the cochlea 140. Other methods of implantation, as well as other stimulation assemblies 118 which adopt a curved configuration, can also be used.

In certain implementations, the stimulation assembly 118 is a non-perimodiolar (e.g., straight) stimulation assembly 118 or a mid-scala assembly which assumes a mid-scala position during or following implantation. Alternatively, the stimulation assembly of certain implementations comprises a short electrode implanted into at least the basal region. The stimulation assembly 118 can extend towards the apical end of the cochlea 140, referred to as the cochlea apex. In certain implementations, the stimulation assembly 118 is configured to be inserted into the cochlea 140 via a cochleostomy. In certain other implementations, a cochleostomy is formed through the oval window 112, the round window 121, the promontory 123, or through an apical turn of the cochlea 140.

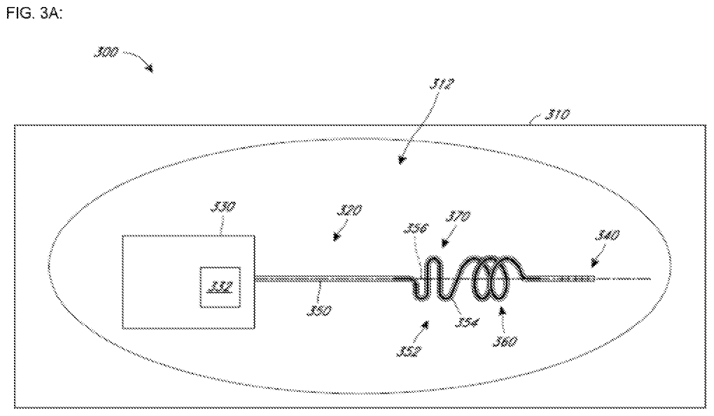

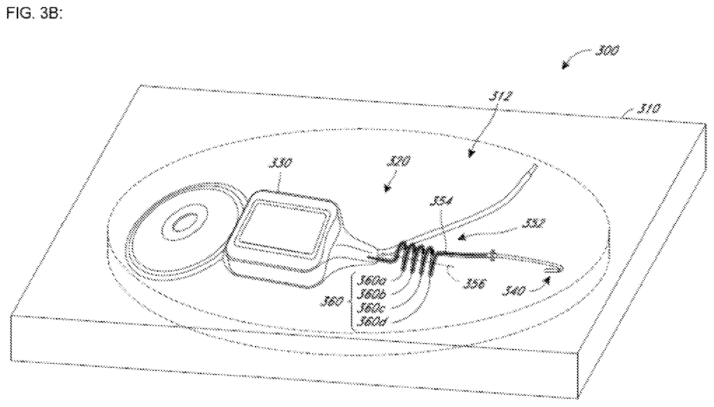

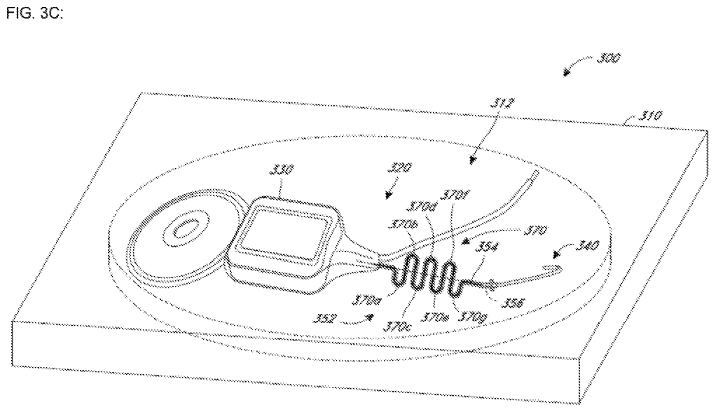

FIG. 3A schematically illustrates an example apparatus 300 in accordance with certain implementations described herein. The apparatus 300 comprises a container 310 comprising a sealed region 312 and a medical device 320 (e.g., medical implant) configured to be implanted on or within the recipient's body. The medical device 320 is contained within the sealed region 312 prior to implantation of the medical device 320. The medical device 320 comprises at least one housing 330 containing circuitry 332, a plurality of signal ports 340 spaced from the at least one housing 330 and configured to be in communication with a portion of the recipient's body, and an elongate assembly 350 extending from the at least one housing 330 and configured to transmit signals between the circuitry 332 and the plurality of signal ports 340. The assembly 350 has at least one portion 352 having a shape while the medical device 320 is within the sealed region 312, the shape comprising at least one loop 360 and/or a plurality of serpentine turns 370.

In certain implementations, the container 310 is configured to protect the medical device 320 during transport of the container 310 and to be opened by a user to access the medical device 320 for implantation. For example, the container 310 can comprise a pouch or a tray and lid. Example materials for the container 310 include, but are not limited to: metal foil, polymer, polyethylene terephthalate glycol (PETG), polyvinyl chloride (PVC), polycarbonate (PC), polypropylene (PP), high impact polystyrene (HIPS), Tyvek® available from Dupont Corp. The container 310 of certain implementations is configured to allow sterilization of the medical device 320 within the sealed region 312 (e.g., by gamma ray irradiation, electron beam irradiation, exposure to ethylene oxide and/or autoclave). For example, at least a portion of the container 310 can comprise a gas-permeable portion configured to allow ethylene oxide into the sealed region 312 for sterilization while preventing ingress of microbes into the sealed region 312. For another example, the sealed region 312 can be hermetically sealed. In certain implementations, the container 310 is compatible with International Organization for Standardization (ISO) 11607-1:2019 (https://www.iso.org/standard/70799.html) for materials and packaging systems intended to maintain sterility of terminally sterilized medical devices until the point of use.

In certain implementations, as illustrated by FIGS. 3B and 3C, the medical device 320 comprises a cochlear implant auditory prosthesis 100. In certain other implementations, the medical device 320 comprise other sensory prostheses (e.g., vestibular devices or implants; visual devices or prostheses), stimulation devices (e.g., cardiac pacemakers or defibrillators; functional electrical stimulation devices) configured to provide stimulation signals to a portion of the recipient's body, or sensor devices configured to sense (e.g., monitor) one or more aspects the recipient's body.

In certain implementations, the at least one housing 330 comprises at least one biocompatible material (e.g., polymer; PEEK; elastomer; silicone; titanium; titanium alloy; ceramic) and the circuitry 332 is hermetically sealed within the at least one housing 330. The circuitry 332 of certain implementations is configured to receive power and/or data from an external source and to control operation of the medical device 320. The at least one housing 330 can comprise a plurality of housing portions, each comprising different portions of the circuitry 332. For example, as schematically illustrated by FIGS. 3B and 3C, the at least one housing 330 and the circuitry 332 can collectively comprise a stimulator/receiver unit, with a first portion of the circuitry 332 comprising at least one internal inductive communication coil 136 configured to receive power and/or data signals from an external inductive communication coil 130) hermetically sealed within a first portion of the at least one housing 330 and a second portion of the circuitry 332 comprising a stimulator unit 120 configured to generate stimulation signals and hermetically sealed within a second portion of the at least one housing 330.

In certain implementations, the plurality of signal ports 340 comprises an array of stimulation elements (e.g., electrical electrodes; electrical contacts; optical emitters; optical contacts) configured to be in communication (e.g., electrical communication; optical communication) with the portion of the recipient's body. For example, the signal ports 340 can comprise a longitudinally aligned and distally extending array 146 of twenty-two (22) stimulation elements 148 (e.g., electrodes) longitudinally spaced from one another along a length of the stimulation assembly 118 and configured to apply electrical stimulation signals to the recipient's cochlea 140. For another example, the signal ports 340 can comprise electrodes configured to receive electrical signals from the portion of the recipient's body.

In certain implementations, the elongate assembly 350 is in mechanical communication with the at least one housing 330 and comprises a plurality of signal conduits (e.g., electrically conductive wires; optical fibers or waveguides) in communication with the circuitry 332 and with the plurality of signal ports 340. In certain implementations, a proximal portion of the assembly 350 is attached to the at least one housing 330, while in certain other implementations, the proximal portion of the assembly 350 and the at least one housing 350 are parts of a single (e.g., unitary; integrated) body. In certain implementations, the signal ports 340 are on or integrated into a distal portion of the assembly 350 as schematically illustrated by FIGS. 3A-3C, while in certain other implementations, the distal portion of the assembly 350 is in mechanical communication with another component of the medical device 320 that contains the signal ports 340.

The signal conduits of certain implementations are configured to transmit signals between the circuitry 332 and the signal ports 340 (e.g., stimulation signals transmitted from the circuitry 332 to the signal ports 340; sensor signals transmitted from the signal ports 340 to the circuitry 332). For example, the assembly 350 can comprise a stimulation assembly 118 having a lead region 220 comprising a body 226 (e.g., silicone) in which the signal conduits are embedded or contained (e.g., tube that is backfilled with silicone or is not backfilled with silicone), and with each of the signal conduits connected to a corresponding one of the signal ports 340 (e.g., stimulation elements 148 of the array 146). In certain implementations, the signal conduits are helixed about the longitudinal direction 221 of the lead region 220, while in certain other implementations, the signal conduits are substantially parallel to the longitudinal direction 221 of the lead region 220 (e.g., arranged in a straight bundle). In certain implementations in which the signal conduits are contained within a tube that is not backfilled with silicone, the elastic stiffness of the lead region 220 is reduced, and the tendency of the lead region 220 to spring back following the user re-shaping the lead region 220 is reduced (e.g., minimized).

In certain implementations, the at least one portion 352 of the assembly 350 comprises at least one malleable element 354 (e.g., wire) on or within the assembly 350. For example, the at least one malleable element 354 can be embedded or contained within a body (e.g., body 226) of the at least one portion 352, as schematically illustrated by FIGS. 3A-3C. The at least one malleable element 354 of certain implementations comprises at least one material selected from the group consisting of: platinum; palladium; ruthenium; rhodium; osmium; iridium; titanium; gold; alloys of one or more of the foregoing; stainless steel; plastically deformable polymer. In certain implementations in which the at least one malleable element 354 comprises electrically conductive metal wire, the at least one malleable element 354 is electrically isolated from the circuitry 332, the signal conduits, and the signal ports 340.

In certain implementations, the at least one malleable element 354 comprises a unitary element (e.g., a single, continuous length of wire), while in certain other implementations, the at least one malleable element 354 comprises a plurality of elements (e.g., two, three, four, or more lengths of wire) that are positioned next to one another (e.g., end-to-end; side-to-side; adjacent elements touching one another, overlapping one another, or spaced from one another). In certain implementations, the at least one malleable element 354 extends longitudinally along only a portion of the assembly 350 (e.g., along the longitudinal direction 221 within the lead region 220, the first portion 222, and/or the second portion 224). For example, the at least one malleable element 354 can extend a length in a range of 10 millimeters to 80 millimeters (e.g., in a range of 15 millimeters to 50 millimeters) along the assembly 350 (e.g., along a longitudinal axis 356 of the at least one portion 352; along the longitudinal direction 221 within the lead region 220, the first portion 22, and/or the second portion 224). In certain implementations, the at least one malleable element 354 is spaced from the housing 330 (e.g., as schematically illustrated by FIG. 3A), while in certain other implementations, the at least one malleable element 354 extends partially into the housing 330 (e.g., as schematically illustrated by FIGS. 3B and 3C). The at least one malleable element 354 can be spaced from the extra-cochlear region 210 or can extend partially into the extra-cochlear region 210.

In certain implementations, the at least one portion 352 is sufficiently stiff and/or otherwise configured to maintain its shape during transport of the container 310 and to allow a user to selectively modify (e.g., extend and/or bend) the shape after removal of the medical device 320 from the container 310 and before or during implantation of the medical device 320. The pre-formed shape (e.g., shape immediately prior to manipulation by the user) and malleability of the at least one portion 352 of certain implementations are configured to allow the assembly 350 to be extended and/or bent with minimal resistance during implantation (e.g., during insertion of the intra-cochlear region 212 into the cochlea 140 by the user). The at least one portion 352 of certain implementations is sufficiently stiff and/or otherwise configured to maintain its user-modified shape upon implantation of the medical device 320 (e.g., the lead region 220 maintains its user-modified shape once the intra-cochlear region 212 is inserted by the user to its final position and is released by the user).

In certain implementations, the width of the at least one malleable element 354 is sufficiently large, in view of the material properties of the malleable element 354, to withstand (e.g., not be altered by; not be malleably shaped by) inertial forces or forces applied by the container 310 to the at least one portion 352 during transport of the container 310. The width of the at least one malleable element 354 of certain implementations is sufficiently small to be controllably malleably shaped (e.g., extended and/or bent) by the user during the implantation process (e.g., using forces applied by the user's fingers or by tweezers held by the user) and is sufficiently large to withstand (e.g., not be altered by; not be malleably shaped by) and/or to counteract restoring forces generated by the elasticity of other components of the assembly 350 (e.g., the signal conduits; the body 226) such that the at least one portion 352 and the at least one malleable element 354 retain their user-modified shape without springing back. For example, the at least one malleable element 354 can have a width (e.g., outer diameter) that is in a range of 0.1 millimeter to 0.3 millimeter (e.g., in a range of 0.15 millimeter to 0.25 millimeter) and/or that is at least ten times greater than a width of any of the individual signal conduits in communication with the circuitry 332 and the signal ports 340.

In certain implementations, as schematically illustrated by FIGS. 3A-3C, the shape of the at least one portion 352 of the assembly 350 comprises a pre-formed coil shape comprising a number of coil turns (e.g., at least one helical coil turn or loop 360 and/or two or more serpentine coil turns 370). For example, as schematically illustrated by FIGS. 3A-3C, due to the at least one loop 360 and/or the plurality of serpentine turns 370, the longitudinal direction 221 (e.g., within the lead region 220, the first portion 222, and/or the second portion 224) deviates from the longitudinal axis 356 of the at least one portion 352.

The at least one loop 360 of certain implementations comprises a single loop 360 (e.g., a single helical coil turn) or a plurality of loops 360 (e.g., two, three, four, or more; helix loops; spiral loops; helical coil turns), and each loop 360 of the at least one loop 360 subtends an angle of at least 360 degrees and has a radius of curvature in a range of 1 millimeters to 15 millimeters. For example, as schematically illustrated by FIG. 3B, the at least one loop 360 comprises four loops 360a-d that are substantially parallel to one another (e.g., each loop 360a-d is substantially planar in a corresponding plane that is substantially perpendicular to the longitudinal axis 356 of the at least one portion 352 of the assembly 350).

The plurality of serpentine turns 370 of certain implementations comprises two or more turns 370 (e.g., two, three, four, or more; sinusoidal turns; semi-circular turns; rectangular turns; triangular turns; S-shaped turns; zig-zag turns), and each turn 370 of the plurality of serpentine turns 370 subtends an angle in a range of 90 degrees to 225 degrees (e.g., in a range of 135 degrees to 225 degrees; approximately 180 degrees) and has a radius of curvature in a range of 1 millimeters to 15 millimeters. In certain implementations, at least two serpentine turns 370 of the plurality of serpentine turns 370 are substantially co-planar. For example, as schematically illustrated by FIG. 3C, the at least one portion 352 has a serpentine coil shape and the plurality of serpentine coil turns 370 comprises seven coil turns 370a-g that are substantially co-planar (e.g., in a common plane that includes the longitudinal axis 356 of the at least one portion 352 of the assembly 350).

In certain implementations, the shape of the at least one portion 352 of the assembly 350 comprises a combination of one or more loops 360 and one or more serpentine turns 370. For example, as schematically illustrated by FIG. 3A, the shape of the at least one portion 352 comprises a plurality of loops 360 and a plurality of serpentine turns 370. While FIG. 3A shows the loops 360 and the turns sequential to one another with the loops 360 closer to the signal ports 340 than are the turns 370, other configurations of the loops 360 and turns 370 are also compatible with certain implementations described herein (e.g., the turns 370 closer to the signal ports 340 than are the loops 360; at least some of the loops 360 alternating with at least some of the turns 370 along the longitudinal axis 356 of the at least one portion 352).

In certain implementations, the assembly 350 has an overall length (e.g., distance from the housing 330 to the signal port 340 closest to the housing 350; distance from the stimulator/receiver unit to the intra-cochlear region 212) while the medical device 320 is within the sealed region 312 (e.g., before implantation) that is approximately equal to a minimum anticipated distance from the anticipated implantation position of the housing 330 (e.g., stimulator/receiver unit) to the portion of the recipient's body (e.g., cochlea 140) at which the signal ports 340 are to be implanted. In certain such implementations, the overall length and shape of the assembly 350 of the medical device 320 removed from the container 310 is configured to facilitate reduced amounts of manipulation and/or adjustment of the assembly 350 (e.g., stimulation assembly 118) by the user immediately prior and/or during implantation of the medical device 320, as compared to the amounts of manipulation and/or adjustment by the user for implantation of conventional medical devices 320. For example, for a medical device 320 comprising a cochlear implant, the overall length and shape of the lead region 220 of the stimulation assembly 118 can be sufficiently small to fit within the mastoid cavity with minimal contact of the lead region 220 with surfaces within the mastoid cavity.

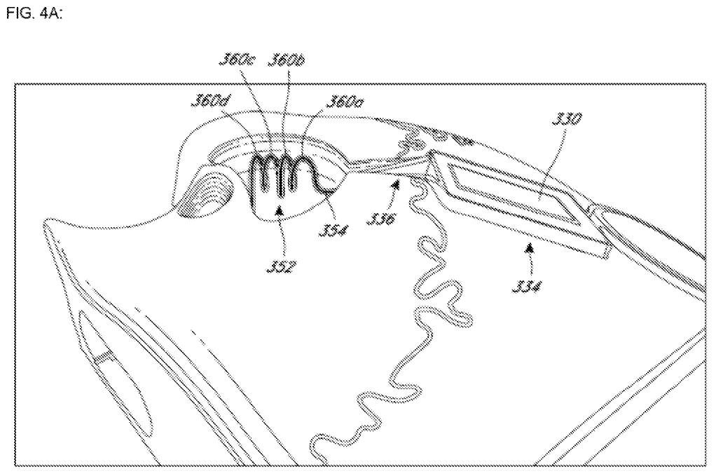

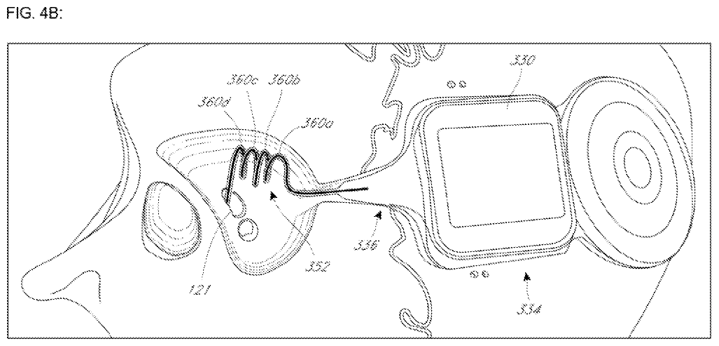

FIGS. 4A and 4B schematically illustrate a perspective view and a top view, respectively, of the example medical device 320 of FIG. 3B on an anatomical mock-up to show the medical device 320 after implantation in accordance with certain implementations described herein. The medical device 320 of FIGS. 4A and 4B is shown after having been removed from the container 310 and after the shape of the at least one portion 352 has been selectively modified by the user before and/or during the implantation. A first portion 334 of the at least one housing 330 of the medical device 320 is positioned on an outer surface of the recipient's skull (e.g., in a recess made by the user during surgery) and a second portion 336 of the at least one housing 330 extends (e.g., into a channel made by the user during surgery) towards the mastoid cavity. The assembly 350 extends from the second portion 336 of the housing 330 into the mastoid cavity with the plurality of signal ports 340 inserted into the cochlea 140 (e.g., via the round window 121). As schematically illustrated by FIGS. 4A and 4B, the assembly 350 and/or the second portion 336 of the at least one housing 330 can have a downward bent relative to the first portion 334 of the at least one housing 330, the downward bent configured to keep the assembly 350 within the mastoid cavity and to prevent the assembly 350 from extending above the outer surface of the skull.

In certain implementations, the at least one portion 352 has a pre-formed coil shape with a first number of coil turns greater than or equal to one (e.g., while the medical device 320 is within the container 310; after removal of the medical device 320 from the container 310 but before implantation). The coil turns can comprise helical coil turns (e.g., of a helical coil), serpentine coil turns (e.g., of a serpentine coil), or a combination thereof. For example, the pre-formed coil shape of the at least one portion 352 schematically illustrated in FIG. 3B has four helical coil turns 360a-d and the pre-formed coil shape of the at least one portion 352 schematically illustrated in FIG. 3C has seven serpentine coil turns 370a-g.

In certain implementations, as schematically illustrated by FIGS. 4A and 4B, the at least one portion 352 of the assembly 350 during and/or after implantation has a modified coil shape with a second number of coil turns that is equal to the first number of coil turns. For example, the modified coil shape of the at least one portion 352 schematically illustrated in FIGS. 4A and 4B has four helical coil turns 360a-d, which is the same number of coil turns as the at least one portion 352 has prior to implantation as shown in FIG. 3B. In certain implementations, the coil turns of the modified coil shape are selectively (e.g., controllably) modified (e.g., bent; twisted; expanded; contracted) by the user while the number of coil turns remains unchanged. For example, as compared to the pre-formed coil shape, the modified coil shape can include one or more modifications (e.g., distortions) while maintaining the same number of coil turns in the modified coil shape, the one or more modifications selected from the group consisting of: modified (e.g., larger or smaller) spacing between adjacent coil turns (e.g., along the longitudinal axis 356 of the at least one portion 352); modified (e.g., larger or smaller) radius of curvature of one or more coil turns; modified (e.g., larger or smaller) angle between planes of adjacent coil turns (e.g., modified from being parallel to non-parallel, or vice versa).

Other modifications that result in a modified coil shape that maintains the character of the pre-formed coil shape (e.g., maintain the same number of loops and/or the same number of serpentine turns) are also compatible with certain implementations described herein. For example, a straight section of the pre-formed coil shape (see, e.g., FIG. 3B) can be selectively (e.g., controllably) modified (e.g., bent) by the user to form at least one bent section in the modified coil shape (see, e.g., FIGS. 4A and 4B), the at least one bent section having an angle less than 180 degrees (e.g., less than 135 degrees; less than 90 degrees). The at least one bent section can be configured to keep the at least one portion 352 away from the user's line of sight during movement and insertion of the plurality of signal ports 340 into the cochlea 140.

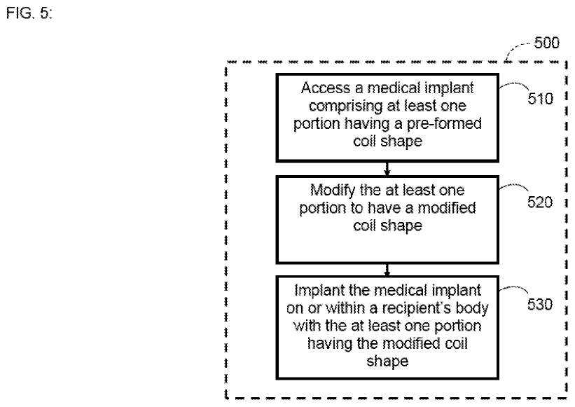

FIG. 5 is a flow diagram of an example method 500 in accordance with certain implementations described herein. In an operational block 510, the method 500 comprises accessing a medical implant (e.g., medical device 320) comprising at least one portion (e.g., at least one portion 352) having a pre-formed coil shape with a first number of coil turns, the first number greater than or equal to one. The medical implant can have the pre-formed coil shape while the medical implant is within a protective container (e.g., container 310) and after removal of the medical implant from the protective container but before implantation. The coil turns of the pre-formed coil shape can comprise helical coil turns (e.g., of a helical coil), serpentine coil turns (e.g., of a serpentine coil), or a combination thereof. For example, accessing the medical implant can comprise opening a sealed container containing the medical implant with the pre-formed coil shape and removing the medical implant from the container.

In an operational block 520, the method 500 further comprises modifying the at least one portion to have a modified coil shape with a second number of coil turns, the second number equal to the first number. For example, modifying the at least one portion can comprise extending and/or bending at least one coil turn of the at least one portion. For example, the user (e.g., surgeon) can remove the medical implant from the packaging, perform no adjustment to the coil shape, insert the electrodes into the cochlea, by which the motion would extend the coil shape by increasing the spacing between the coils, and then the user releases his/her grip on the medical implant. If there is no spring-back, the user can opt to not make further adjustment to the coil shape. The user can opt to make some final adjustment to the coil shape or position after insertion.

In an operational block 530, the method 500 further comprises implanting the medical implant on or within a recipient's body with the at least one portion having the modified coil shape. For example, the medical implant can comprise a cochlear implant comprising a stimulation unit and the at least one portion can extend from the stimulation unit and comprise a plurality of electrodes, and implanting the medical implant can comprise implanting the stimulation unit on and/or in the skull of the recipient, extending the at least one portion within a mastoid cavity of the recipient, and inserting at least a portion of the plurality of electrodes into a cochlea of the recipient.

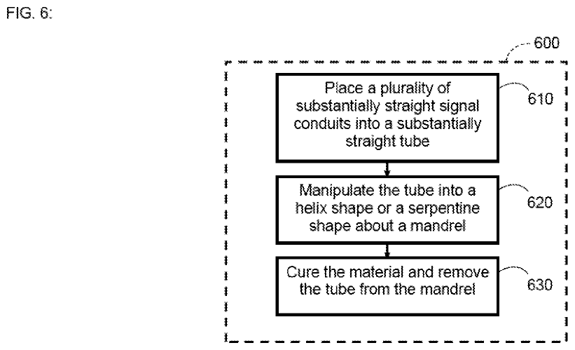

In certain implementations, the pre-formed coil shape is configured to facilitate the fabrication of the assembly 350 as compared to existing manufacturing processes in which the signal conduits are helixed about a substantially straight longitudinal direction 221 and then sealed within silicone. FIG. 6 is a flow diagram of an example method 600 in accordance with certain implementations described herein. In an operational block 610, the method 600 comprises placing (e.g., inserting) a plurality of substantially straight signal conduits (e.g., electrical conduits; electrical wires; optical conduits; optical fibers) into a substantially straight tube (e.g., silicone) and inserting a material (e.g., polymer; PEEK; elastomer; polyurethane; silicone) into the tube. In an operational block 620, the method 600 further comprises manipulating the tube containing the plurality of signal conduits and the material into a helix shape or a serpentine shape about a mandrel. For example, the mandrel can comprise a cylindrical shape having a radius of curvature (e.g., in a range of 1 millimeters to 15 millimeters) and the tube containing the signal conduits can be wrapped around the mandrel to form a series of helical turns about the longitudinal axis 356. For another example, the mandrel can comprise a plurality of protrusions (e.g., pins) and the tube containing the signal conduits can be bent around the protrusions to form a series of serpentine turns (e.g., co-planar with the longitudinal axis 356). In an operational block 630, the method 600 further comprises curing the material and removing the tube from the mandrel. In certain implementations, the method further comprises inserting a malleable element on or within the tube prior to said manipulating the tube.

In certain implementations, the pre-formed coil shape is configured to reduce a mechanical resistance of the at least one portion 352 to bending and/or twisting, as compared to a medical device 320 without the pre-formed coil shape, thereby reducing a risk of the signal ports 340 at a distal end of the assembly 350 moving and/or twisting following insertion into the recipient's body (e.g., into the cochlea 140) and of a concomitant loss of functionality (e.g., reduction of a hearing precept provided to the recipient). In certain implementations, the pre-formed coil shape is configured to facilitate user-controlled modifications of the at least one portion 352 (e.g., lengthening or contracting the at least one portion 352 with minimal mechanical resistance) into a modified coil shape that more easily allows for variation in anatomy or user preference for implantation and/or accommodates skull growth (e.g., for pediatric recipients) without residual tension. For example, the at least one malleable element 354 and the pre-formed coil shape can streamline the implantation process by facilitating the user (e.g., surgeon) placing the medical device 320 in the periosteal pocket, inserting the electrodes 148 of the stimulation assembly 118 into the cochlea 140, and then releasing the stimulation assembly 118 without need for further manipulation since the stimulation assembly 118 is sufficiently stiff to hold itself in position and is already confined within the mastoid cavity.

In certain implementations, the stiffness of the at least one portion 352 of the assembly 350 is configured to stabilize the position of the signal ports 340, thereby reducing (e.g., minimizing) post-placement movement of the signal ports 340. For example, the stiffness of the stimulation assembly 118 having the modified coil shape can reduce post-insertion movement of the array 146 in the cochlea 140 which could otherwise occur during securing of a conventional stimulation assembly 118, thereby reducing (e.g., minimizing) the risk of post-surgical migration, additional trauma, and the concomitant loss of acoustic hearing. The at least one malleable element 354 of certain implementations can counteract residual elastic energy within the assembly 350 which could otherwise cause erosion of tissue over time or spontaneous post-surgical movement of the signal ports 340.

In certain implementations, the pre-formed coil shape is configured to reduce (e.g., eliminate) the need to directly manipulate (e.g., manually; with tweezers) the at least one portion 352 or other portions of the assembly 350 to form complex shapes, thereby reducing the risk of damage to the medical device 320 by the user prior to and/or during implantation. For example, to form a substantially straight stimulation assembly 118 into a desired modified shape can require the use of both hands by the user (e.g., surgeon) and, when performed after electrode insertion, can risk unwanted movement of the array 146 in the cochlea 140. By having the pre-formed coil shape, the medical device 320 of certain implementations is configured to simplify the implantation process and to facilitate implantation without the user applying potentially excessive stresses (e.g., stresses which can damage the signal conduits) to an assembly having a substantially straight pre-implantation shape.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a container comprising a sealed region; and
a medical device configured to be implanted on or within the recipient's body, the medical device contained within the sealed region prior to implantation of the medical device, the medical device comprising:
at least one housing containing circuitry;
a plurality of signal ports spaced from the at least one housing, the plurality of signal ports configured to be in communication with a portion of the recipient's body; and
an elongate assembly extending from the at least one housing and configured to transmit signals between the circuitry and the plurality of signal ports, the assembly having at least one portion having a shape while the medical device is within the sealed region, the shape comprising at least one loop and/or a plurality of serpentine turns.

2. The apparatus of claim 1, wherein the at least one portion comprises at least one malleable element configured to maintain the shape of the at least one portion during transport of the apparatus, to allow for selective modification of the shape by a user before or during implantation of the medical device, and to maintain the user-modified shape after implantation.

3. The apparatus of claim 1, wherein the container is configured to protect the medical device during transport of the container and to be opened by a user to access the medical device for implantation.

4. The apparatus of claim 1, wherein the at least one portion is configured to maintain the shape during transport of the container and to allow a user to modify the shape after removal of the medical device from the container and before or during implantation of the medical device.

5. The apparatus of claim 1, wherein the shape comprises at least one loop that subtends an angle of at least 360 degrees and is substantially planar in a plane substantially perpendicular to a longitudinal axis of the at least one portion of the assembly.

6. The apparatus of claim 5, wherein the at least one loop comprises a plurality of loops substantially parallel to one another.

7. The apparatus of claim 5, wherein the at least one loop has a radius of curvature in a range of 1 millimeter to 15 millimeters.

8. The apparatus of claim 1, wherein at least two serpentine turns of the plurality of serpentine turns are substantially co-planar.

9. The apparatus of claim 8, wherein each of the at least two serpentine turns have a radius of curvature in a range of 1 millimeter to 15 millimeters.

10. The method of claim 1, wherein the medical device comprises a cochlear implant, the at least one housing comprises a stimulation unit, the assembly comprises a stimulation assembly extending from the stimulation unit, and the plurality of signal ports comprises a plurality of electrodes configured to be inserted into a cochlea of the recipient.

11. A method comprising:
accessing a medical implant comprising at least one portion having a pre-formed coil shape with a first number of coil turns, the first number greater than or equal to one;
modifying the at least one portion to have a modified coil shape with a second number of coil turns, the second number equal to the first number; and
implanting the medical implant on or within a recipient's body with the at least one portion having the modified coil shape.

12. The method of claim 11, wherein the pre-formed coil shape comprises a helical coil and the coil turns are helical coil turns.

13. The method of claim 11, wherein the pre-formed coil shape comprises a serpentine coil and the turns are serpentine coil turns.

14. The method of claim 11, wherein accessing the medical implant comprising opening a sealed container containing the medical implant with the pre-formed coil shape and removing the medical implant from the container.

15. The method of claim 11, wherein modifying the at least one portion comprises extending and/or bending at least one coil turn of the at least one portion.

16. The method of claim 11, wherein the medical implant comprises a cochlear implant comprising a stimulation unit and the at least one portion extends from the stimulation unit and comprises a plurality of electrodes, wherein implanting the medical implant comprises implanting the stimulation unit on and/or in the skull of the recipient, extending the at least one portion within a mastoid cavity of the recipient, and inserting at least a portion of the plurality of electrodes into a cochlea of the recipient.

17. A method comprising:
placing a plurality of substantially straight signal conduits into a substantially straight tube and inserting a material into the tube;
manipulating the tube containing the plurality of signal conduits and the material into a helix shape or a serpentine shape about a mandrel; and
curing the material and removing the tube from the mandrel.

18. The method of claim 17, wherein the tube comprises silicone.

19. The method of claim 17, wherein the material comprises at least one of: polymer, PEEK, elastomer, polyurethane, and silicone.

20. The method of claim 17, wherein the mandrel comprises a cylindrical shape having a radius of curvature in a range of 1 millimeter to 15 millimeters.

21. The method of claim 17, further comprising inserting a malleable element on or within the tube prior to said manipulating the tube.
